(19) United States
(12) Patent Application Publication
(10) Pub. No.: US 2025/0140416 A1
(43) Pub. Date: May 1, 2025

(54) DYNAMIC PRECISION TIME SERIES ENSEMBLE SELECTION

(71) Applicant: Optum, Inc., Minnetonka, MN (US)

(72) Inventors: Danita Kiser, Marietta, GA (US); Laveena Garg, Gurugram (IN); Madhuri Yadav, Gurugram (IN); Lajja N. Pancholy, San Diego, CA (US); Amit K. Meher, Bengaluru (IN); Jyoti Nahata, Delhi (IN)

(21) Appl. No.: 18/494,446

(22) Filed: Oct. 25, 2023

(51) Int. Cl. G16H 50/30; G06N 20/20

(52) U.S. Cl. CPC G16H 50/30; G06N 20/20

(57) ABSTRACT

Embodiments provide for generating dynamic precision time series ensembles of machine learning models according to ensemble levels and a validation period, selecting a top performing scheme combination object for each time stamp in a test period, generating a prediction based on the top performing scheme combination object, and initiating the performance of one or more prediction-based actions based on the prediction.

### BACKGROUND

The increased emergence of novel infectious diseases and transfer of existing infectious pathogens requires swift and accurate predictions of the course of a disease. Instead of depending solely on individual forecasts that leverage one technique or algorithm, scientists often bundle them together to create an ensemble of forecast models. These ensembles better predict long-term trends in outbreaks, hospitalizations, and deaths. However, recent infectious disease spread (e.g., pandemic) proved that these same ensembles fail to provide precise enough forecasts for short-term trends.

Through applied effort, ingenuity, and innovation, many of these identified deficiencies and problems have been solved by developing solutions that are structured in accordance with embodiments of the present disclosure, many examples of which are described in detail herein.

### BRIEF SUMMARY

Embodiments provide for dynamic precision time series ensemble selection.

An example system comprises memory and one or more processors communicatively coupled to the memory, the one or more processors configured to generate one or more ensembles of machine learning models according to one or more weighting schemes, wherein the machine learning models are selected from a plurality of machine learning models configured to predict a future risk of infectious diseases by location; generate one or more scheme combination objects according to a validation period, wherein the one or more scheme combination objects comprise one or more machine learning models to which a particular weighting scheme has been applied; select, based on an ensemble level and one or more performance metrics, a top performing scheme combination object for each time stamp in a test period; generate a prediction based on the top performing scheme combination object; and initiate the performance of one or more prediction-based actions based on the prediction.

An example of one or more non-transitory computer-readable storage media includes instructions that, when executed by one or more processors, cause the one or more processors to generate one or more ensembles of machine learning models according to one or more weighting schemes, wherein the machine learning models are selected from a plurality of machine learning models configured to predict a future risk of infectious diseases by location; generate one or more scheme combination objects according to a validation period, wherein the one or more scheme combination objects comprise one or more machine learning models to which a particular weighting scheme has been applied; select, based on an ensemble level and one or more performance metrics, a top performing scheme combination object for each time stamp in a test period; generate a prediction based on the top performing scheme combination object; and initiate the performance of one or more prediction-based actions based on the prediction.

An example computer-implemented method includes generating, by one or more processors, one or more ensembles of machine learning models according to one or more weighting schemes, wherein the machine learning models are selected from a plurality of machine learning models configured to predict a future risk of infectious diseases by location; generating, by the one or more processors, one or more scheme combination objects according to a validation period, wherein the one or more scheme combination objects comprise one or more machine learning models to which a particular weighting scheme has been applied; selecting, by the one or more processors and based on an ensemble level and one or more performance metrics, a top performing scheme combination object for each time stamp in a test period; generating, by the one or more processors, a prediction based on the top performing scheme combination object; and initiating, by the one or more processors, the performance of one or more prediction-based actions based on the prediction.

The above summary is provided merely for purposes of summarizing some example embodiments to provide a basic understanding of some aspects of the present disclosure. Accordingly, it will be appreciated that the above-described embodiments are merely examples and should not be construed to narrow the scope or the spirit of the present disclosure in any way. It will be appreciated that the scope of the present disclosure encompasses many potential embodiments in addition to those here summarized, some of which will be further described below. Other features, aspects, and advantages of the subject matter will become apparent from the description, the drawings, and the claims.

### BRIEF DESCRIPTION OF THE DRAWINGS

Having thus described certain example embodiments of the present disclosure in general terms above, non-limiting and non-exhaustive embodiments of the subject disclosure will now be described with reference to the accompanying drawings which are not necessarily drawn to scale. The components illustrated in the accompanying drawings may or may not be present in certain embodiments described herein. Some embodiments may include fewer (or more) components than those shown in the drawings. Some embodiments may include the components arranged in a different way:

FIG. 1 provides an example overview of a system that can be used to practice embodiments of the present disclosure.

FIG. 2 provides an example predictive analysis computing entity in accordance with some embodiments discussed herein.

FIG. 3 provides an example external computing entity in accordance with some embodiments discussed herein.

FIGS. 4A, 4B, and 4C provide an example data flow architecture in accordance with some embodiments discussed herein.

FIG. 5 provides example operations for performance in accordance with some embodiments discussed herein.

FIG. 6 provides examples of operator configurable validation periods associated with some embodiments discussed herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not indicate being based only on or based solely on the referenced element or elements unless so indicated. Like numbers refer to like elements throughout. Moreover, while certain embodiments of the present disclosure are described with reference to schema matching, one of ordinary skill in the art will recognize that the disclosed concepts can be used to perform other types of data analysis.

### I. COMPUTER PROGRAM PRODUCTS, METHODS, AND COMPUTING ENTITIES

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as in a particular directory, folder, or library. Software components may be static (e.g., pre-established, or fixed) or dynamic (e.g., created or modified at the time of execution).

A computer program product may include non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatuses, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### II. EXAMPLE FRAMEWORK

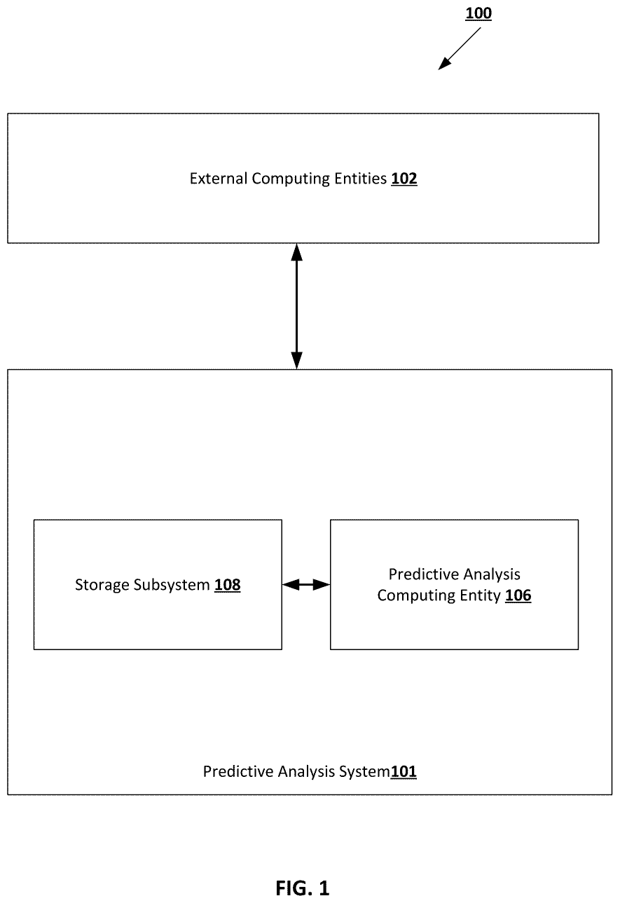

FIG. 1 provides an example overview of an architecture that can be used to practice embodiments of the present disclosure. The architecture 100 may include a predictive analysis system 101 and a predictive analysis computing entity 106 configured to generate outputs that can be used to perform one or more output-based actions. The predictive analysis system 101 may communicate with one or more external computing entities 102A-N using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (e.g., network routers, and/or the like).

The architecture 100 includes a storage subsystem 108 configured to store at least a portion of the data utilized by the predictive analysis system 101. The predictive analysis computing entity 106 may be in communication with the external computing entities 102A-N. The predictive analysis computing entity 106 may be configured to: (i) train one or more machine learning models based on a training data store stored in the storage subsystem 108, (ii) store trained machine learning models as part of a model definition data store of the storage subsystem 108, (iii) utilize trained machine learning models to perform an action, (iv) and/or the like.

In one example, the predictive analysis computing entity 106 may be configured to generate an embedding, prediction, classification, and/or any other data insight based on data provided by an external computing entity such as external computing entity 102A, external computing entity 102B, and/or the like.

The storage subsystem 108 may be configured to store the model definition data store and the training data store for one or more machine learning models generated or utilized herein. The predictive analysis computing entity 106 may be configured to receive requests and/or data from at least one of the external computing entities 102A-N, process the requests and/or data to generate outputs (e.g., predictive outputs, classification outputs, and/or the like), and provide the outputs to at least one of the external computing entities 102A-N. In some embodiments, the external computing entity 102A, for example, may periodically update/provide raw and/or processed input data to the predictive analysis system 101. The external computing entities 102A-N may further generate user interface data (e.g., one or more data objects) corresponding to the outputs and may provide (e.g., transmit, send, and/or the like) the user interface data corresponding with the outputs for presentation to the external computing entity 102A (e.g., to an end-user).

The storage subsystem 108 may be configured to store at least a portion of the data utilized by the predictive analysis computing entity 106 to perform one or more steps/operations and/or tasks described herein. The storage subsystem 108 may be configured to store at least a portion of operational data and/or operational configuration data including operational instructions and parameters utilized by the predictive analysis computing entity 106 to perform the one or more steps/operations described herein. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

The predictive analysis computing entity 106 can include an analysis engine and/or a training engine. The analysis engine may be configured to perform one or more data analysis techniques. The training engine may be configured to train and/or retrain one or more machine learning models.

### Example Predictive Analysis Computing Entity

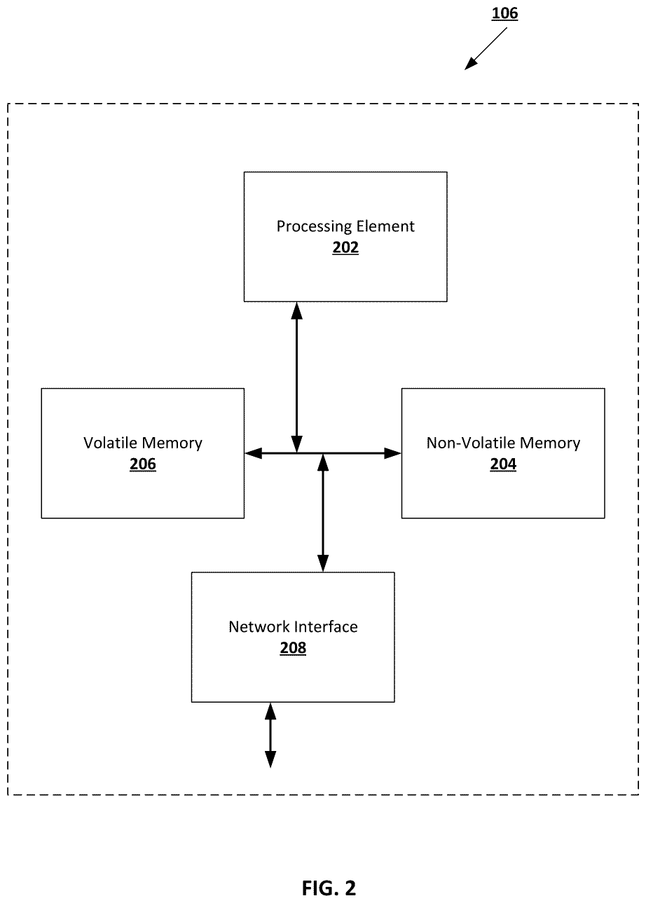

FIG. 2 provides an example predictive analysis computing entity 106 in accordance with some embodiments discussed herein. In general, the terms computing entity, computer, entity, device, system, and/or similar words used herein interchangeably may refer to, for example, one or more computers, computing entities, desktops, mobile phones, tablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, steps/operations, and/or processes described herein. Such functions, steps/operations, and/or processes may include, for example, transmitting, receiving, operating on, processing, displaying, storing, determining, creating/generating, monitoring, evaluating, comparing, and/or similar terms used herein interchangeably. In one embodiment, these functions, steps/operations, and/or processes can be performed on data, content, information, and/or similar terms used herein interchangeably.

The predictive analysis computing entity 106 may include a network interface 208 for communicating with various computing entities, such as by communicating data, content, information, and/or similar terms used herein interchangeably that can be transmitted, received, operated on, processed, displayed, stored, and/or the like.

In one embodiment, the predictive analysis computing entity 106 may include or be in communication with a processing element 202 (also referred to as processors, processing circuitry, and/or similar terms used herein interchangeably) that communicate with other elements within the predictive analysis computing entity 106 via a bus, for example. As will be understood, the processing element 202 may be embodied in a number of different ways including, for example, as at least one processor/processing apparatus, one or more processors/processing apparatuses, and/or the like.

For example, the processing element 202 may be embodied as one or more complex programmable logic devices (CPLDs), microprocessors, multi-core processors, coprocessing entities, application-specific instruction-set processors (ASIPs), microcontrollers, and/or controllers. Further, the processing element 202 may be embodied as one or more other processing devices or circuitry. The term circuitry may refer to an entirely hardware embodiment or a combination of hardware and computer program products. Thus, the processing element 202 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 202 may be configured for a particular use or configured to execute instructions stored in one or more memory elements including, for example, one or more volatile memories 206 and/or non-volatile memories 204. As such, whether configured by hardware or computer program products, or by a combination thereof, the processing element 202 may be capable of performing steps or operations according to embodiments of the present disclosure when configured accordingly. The processing element 202, for example in combination with the one or more volatile memories 206 and/or or non-volatile memories 204, may be capable of implementing one or more computer-implemented methods described herein. In some implementations, the predictive analysis computing entity 106 can include a computing apparatus, the processing element 202 can include at least one processor of the computing apparatus, and the one or more volatile memories 206 and/or non-volatile memories 204 can include at least one memory including program code. The at least one memory and the program code can be configured to, upon execution by the at least one processor, cause the computing apparatus to perform one or more steps/operations described herein.

The non-volatile memories 204 (also referred to as non-volatile storage, memory, memory storage, memory circuitry, media, and/or similar terms used herein interchangeably) may include at least one non-volatile memory device, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile memories 204 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

The one or more volatile memories (also referred to as volatile storage, memory, memory storage, memory circuitry, media, and/or similar terms used herein interchangeably) can include at least one volatile memory device, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile memories 206 may be used to store at least portions of the databases, database instances, database management systems, data, applications, programs, program modules, scripts, source code, object code, byte code, compiled code, interpreted code, machine code, executable instructions, and/or the like being executed by, for example, the processing element 202. Thus, the databases, database instances, database management systems, data, applications, programs, program modules, scripts, source code, object code, byte code, compiled code, interpreted code, machine code, executable instructions, and/or the like may be used to control certain embodiments of the operation of the predictive analysis computing entity 106 with the assistance of the processing element 202.

As indicated, in one embodiment, the predictive analysis computing entity 106 may also include the network interface 208 for communicating with various computing entities, such as by communicating data, content, information, and/or the like that can be transmitted, received, operated on, processed, displayed, stored, and/or the like. Such communication data may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive analysis computing entity 106 may be configured to communicate via wireless client communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

### Example External Computing Entity

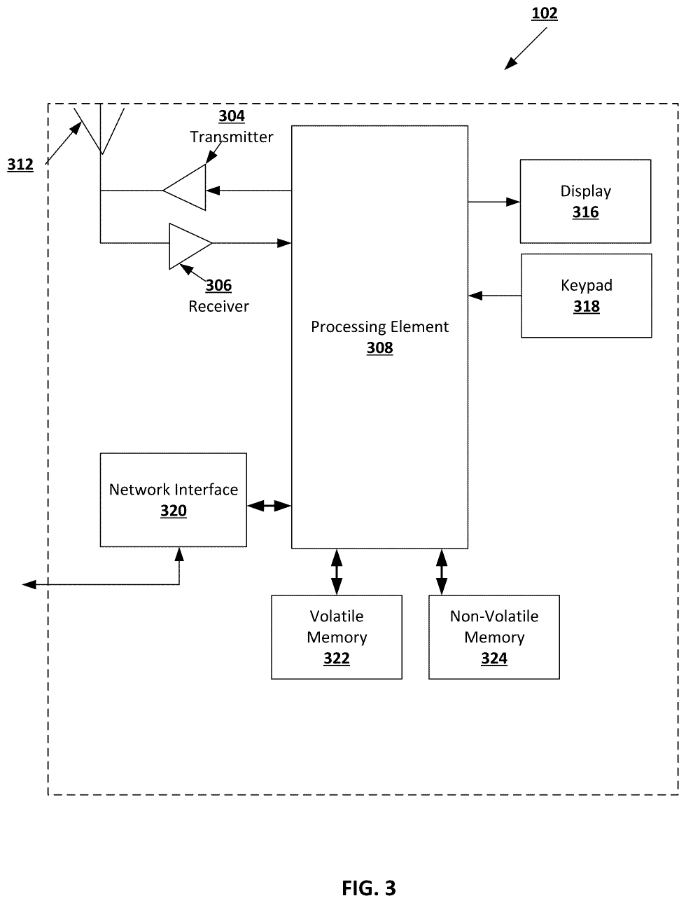

FIG. 3 provides an example external computing entity 102A in accordance with some embodiments discussed herein. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, steps/operations, and/or processes described herein. Various parties can operate the external computing entities 102A-N. As shown in FIG. 3, the external computing entity 102A can include an antenna 312, a transmitter 304 (e.g., radio), a receiver 306 (e.g., radio), and/or an external entity processing element 308 (e.g., CPLDs, microprocessors, multi-core processors, coprocessing entities, ASIPs, microcontrollers, and/or controllers) that provides signals to and receives signals from the transmitter 304 and the receiver 306, correspondingly. As will be understood, the external entity processing element 308 may be embodied in a number of different ways including, for example, as at least one processor/processing apparatus, one or more processors/processing apparatuses, and/or the like as described herein with reference the processing element 202.

The signals provided to and received from the transmitter 304 and the receiver 306, correspondingly, may include signaling information/data in accordance with air interface standards of applicable wireless systems. In this regard, the external computing entity 102A may be capable of operating with one or more air interface standards, communication protocols, modulation types, and access types. More particularly, the external computing entity 102A may operate in accordance with any of a number of wireless communication standards and protocols, such as those described above with regard to the predictive analysis computing entity 106. In a particular embodiment, the external computing entity 102A may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102A may operate in accordance with multiple wired communication standards and protocols, such as those described above with regard to the predictive analysis computing entity 106 via an external entity network interface 320.

Via these communication standards and protocols, the external computing entity 102A can communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 102A can also download changes, add-ons, and updates, for instance, to its firmware, software (e.g., including executable instructions, applications, program modules), operating system, and/or the like.

According to one embodiment, the external computing entity 102A may include location determining embodiments, devices, modules, functionalities, and/or the like. For example, the external computing entity 102A may include outdoor positioning embodiments, such as a location module adapted to acquire, for example, latitude, longitude, altitude, geocode, course, direction, heading, speed, universal time (UTC), date, and/or various other information/data. In one embodiment, the location module can acquire data such as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the DecimalDegrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating a position of the external computing entity 102A in connection with a variety of other systems, including cellular towers, Wi-Fi access points, and/or the like. Similarly, the external computing entity 102A may include indoor positioning embodiments, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning embodiments can be used in a variety of settings to determine the location of someone or something to within inches or centimeters.

The external computing entity 102A may include a user interface 316 (e.g., a display, speaker, and/or the like) that can be coupled to the external entity processing element 308. In addition, or alternatively, the external computing entity 102A can include a user input interface 319 (e.g., keypad, touch screen, microphone, and/or the like) coupled to the external entity processing element 308).

For example, the user interface 316 may be a user application, browser, and/or similar words used herein interchangeably executing on and/or accessible via the external computing entity 102A to interact with and/or cause the display, announcement, and/or the like of information/data to a user. The user input interface 318 can comprise any of a number of input devices or interfaces allowing the external computing entity 102A to receive data including, as examples, a keypad (hard or soft), a touch display, voice/speech interfaces, motion interfaces, and/or any other input device. In embodiments including a keypad, the keypad can include (or cause display of) the conventional numeric (0-9) and related keys (#, *, and/or the like), and other keys used for operating the external computing entity 102A and may include a full set of alphabetic keys or set of keys that may be activated to provide a full set of alphanumeric keys. In addition to providing input, the user input interface 318 can be used, for example, to activate or deactivate certain functions, such as screen savers, sleep modes, and/or the like.

The external computing entity 102A can also include one or more external entity non-volatile memories 322 and/or one or more external entity volatile memories 324, which can be embedded within and/or may be removable from the external computing entity 102A. As will be understood, the external entity non-volatile memories 322 and/or the external entity volatile memories 324 may be embodied in a number of different ways including, for example, as described herein with reference the non-volatile memories 204 and/or the volatile memories 206.

### III. EXAMPLES OF CERTAIN TERMS

In some embodiments, the term “horizon” refers to time points at which predictions are performed. In some embodiments, a horizon can be daily, weekly, operator configurable, and/or the like.

In some embodiments, the term “ensemble level” refers to a level at which an ensemble is evaluated. For example, an ensemble can be evaluated at a location level, a horizon level, and/or a location+horizon level. In embodiments, each ensemble level is evaluated. These levels are evaluated in the test period to determine the optimal level for performance.

In some embodiments, the term “model repository” refers to a repository storing individual model forecasts, which are input to a dynamic precision ensemble.

In some embodiments, the term “performance metrics” refers to measurements representative of model performance. For example, performance metrics may include MAPE (Mean Absolute Percentage Error), RMSE (Root Mean Square Error), MAE (Mean Absolute Error), Interval Accuracies, and/or the like to evaluate performance.

In some embodiments, the term “scheme” refers to a programmatic weighting applied to one or more models of multiple machine learning models of a model bank. In some example embodiments, a scheme is mean, median, performance-based, rank-based, and/or the like.

In some embodiments, the term “combination” refers to a programmatic selection of a combination of one or more models of multiple machine learning models of a model bank. In some examples, a combination can be all individual models of the multiple machine learning models, a top x models (e.g., where x≤X total individual models in the model bank), and/or the like.

In some embodiments, the term “scheme combination object” refers to a selected scheme that is applied to the one or more models of the machine learning models as well as the selected combination of the one or more models. For example, a scheme combination object (e.g., also referred to as Scheme x Combination herein) can represent a performance-based weighting that is applied to the top x models of the multiple machine learning models.

In some embodiments, the term “combination vector” refers to a data structure representative of a combination of predictions for locations in a geospatial region having historical time-series data. For example:
X represents a count (e.g., number) of machine learning models in a model bank;
L represents the locations comprising any geospatial region having historical time-series data (e.g., states, counties, Metropolitan Statistical Areas (MSAs), and the like);
H represents the time-series predictions before and after the current point in time (e.g., horizons);
V represents the validation period used to select the top x (x<=X models in model bank) models and the best ensemble Scheme x Combinations (e.g., scheme combination objects);
T represents the test period where the dynamic precision ensembles are evaluated, and where the optimal ensemble level (Location, Horizon, Location+Horizon) and optimal validation period range are determined;
E represents an ensemble model combination corresponding to a particular scheme (e.g., Eₑ is the mean ensemble of the top x models, Eₑ₊ₙ is the weighted ensemble of the top x models); and
P represents the performance measure for each model, location, and horizon (XₓLₗH₀) or ensemble (e.g., or models), location, and horizon (EₑLₗH₀).

Where H₀ is the current point in time, and Hᵢ (i≤n; n=total #of horizons) denotes the number of time periods before or after the current point in time (e.g., the horizon). A positive i represents a forecast, a negative i represents a back cast, i=0 is a nowcast. In this example, L represents all counties in the United States, X represents the number of models in the model bank (e.g., 401), H represents weeks and E represents any ensemble Scheme x Combination (e.g., scheme combination object).

$$X = {X}_{1} , {X}_{2} , … , {X}_{x}$$

$$L = {L}_{1} , {L}_{2} , ... . , {L}_{l}$$

$$H = {H}_{0} , {H}_{1} , … , {H}_{h}$$

$$E = {E}_{1} , {E}_{2} , … , {E}_{e}$$

An example combination vector X₁ L₃ H₀ represents model 1 (X₁), county 3 (L₃), and the forecast for the current week (e.g., H₀, week 0). Another example combination vector X₂ L₃ H₀ represents model 2 (X₁), county 3 (L₃), and the forecast for the current week (e.g., H₀, week 0). Another example combination vector Xₓ Lₗ Hₕ represents model x (X₁), county 1 (Li), and the forecast for week h (e.g., Hₕ, h or h−1 weeks from a current network time). Another example combination vector EₑL₃H₀ represents ensemble a mean ensemble of the top x models (Eₑ), county 3 (L₃), and the forecast for the current week (H₀).

In some embodiments, the term “validation period” refers to a point of network time or a duration of network time during which one or more models, ensembles, or scheme combination objects are determined to have best performed for the particular selection of other hyperparameters (e.g., location, horizon, etc.). That is, the top x models and/or scheme combination objects are selected for a particular validation period. The validation period can be configurable by a user or operator, and/or the validation period can be selected by the predictive analysis computing entity 106 based on other values under evaluation.

In some embodiments, the term “test period” refers to a point of network time or a duration of network time during which the performance of a dynamic precision ensemble output is compared to the performance of any individual model output or static ensemble output.

In some embodiments, the term “dynamic precision ensemble” refers to predictions or forecasts generated by the predictive analysis computing entity 106 using an optimal ensemble level, validation period, and/or other configurable parameters as described herein.

In some embodiments, the terms “trained machine learning model,” “machine learning model,” “model,” “one or more models,” or “ML” refer to a machine learning or deep learning task or mechanism. Machine learning is a method used to devise complex models and algorithms that lend themselves to prediction. A machine learning model is a computer-implemented algorithm that may learn from data with or without relying on rules-based programming. These models enable reliable, repeatable decisions and results and uncovering of hidden insights through machine-based learning from historical relationships and trends in the data. In some embodiments, the machine learning model is a clustering model, a regression model, a neural network, a random forest, a decision tree model, a classification model, or the like. In some embodiments, “machine learning model” may refer herein to one or more time series models and/or non-classification models.

A machine learning model is initially fit or trained on a training dataset (e.g., a set of examples used to fit the parameters of the model). The model may be trained on the training dataset using supervised or unsupervised learning. The model is run with the training dataset and produces a result, which is then compared with a target, for each input vector in the training dataset. Based on the result of the comparison and the specific learning algorithm being used, the parameters of the model are adjusted. The model fitting may include both variable selection and parameter estimation. Successively, the fitted model is used to predict the responses for the observations in a second dataset called the validation dataset. The validation dataset provides an unbiased evaluation of a model fit on the training dataset while tuning the model's hyperparameters (e.g., the number of hidden units in a neural network). In some embodiments, the model can be trained and/or trained in real-time (e.g., online training) while in use.

The machine learning models as described herein may make use of multiple ML engines, e.g., for analysis, transformation, and other needs. The system may train different ML models for different needs and different ML-based engines. The system may generate new models (based on the gathered training data) and may evaluate their performance against the existing models. Training data may include any of the gathered information, as well as information on actions performed based on the various recommendations.

The ML models may be any suitable model for the task or activity implemented by each ML-based engine. Machine learning models may be some form of neural network. The underlying ML models may be learning models (supervised or unsupervised). As examples, such algorithms may be prediction (e.g., linear regression) algorithms, classification (e.g., decision trees, k-nearest neighbors) algorithms, time-series forecasting (e.g., regression-based) algorithms, association algorithms, clustering algorithms (e.g., K-means clustering, Gaussian mixture models, DBscan), or Bayesian methods (e.g., Naïve Bayes, Bayesian model averaging, Bayesian adaptive trials), image to image models (e.g., FCN, PSPNet, U-Net) sequence to sequence models (e.g., RNNs, LSTMs, BERT, Autoencoders) or Generative models (e.g., GANs).

Alternatively, ML models may implement statistical algorithms, such as dimensionality reduction, hypothesis testing, one-way analysis of variance (ANOVA) testing, principal component analysis, conjoint analysis, neural networks, support vector machines, decision trees (including random forest methods), ensemble methods, and other techniques. Other ML models may be generative models (such as Generative Adversarial Networks or auto-encoders).

In various embodiments, the ML models may undergo a training or learning phase before they are released into a production or runtime phase or may begin operation with models from existing systems or models. During a training or learning phase, the ML models may be tuned to focus on specific variables, to reduce error margins, or to otherwise optimize their performance. The ML models may initially receive input from a wide variety of data, such as the gathered data described herein.

The terms “data,” “content,” “digital content,” “digital content object,” “signal,” “information,” and similar terms may be used interchangeably to refer to data capable of being transmitted, received, and/or stored in accordance with embodiments of the present disclosure. Thus, use of any such terms should not be taken to limit the spirit and scope of embodiments of the present disclosure. Further, where a computing device is described herein to receive data from another computing device, it will be appreciated that the data may be received directly from another computing device or may be received indirectly via one or more intermediary computing devices, such as, for example, one or more servers, relays, routers, network access points, base stations, hosts, and/or the like, sometimes referred to herein as a “network.” Similarly, where a computing device is described herein to send data to another computing device, it will be appreciated that the data may be transmitted directly to another computing device or may be transmitted indirectly via one or more intermediary computing devices, such as, for example, one or more servers, relays, routers, network access points, base stations, hosts, and/or the like

### IV. OVERVIEW, TECHNICAL IMPROVEMENTS, AND TECHNICAL ADVANTAGES

The emergence of novel infectious diseases has increased significantly in recent decades. The transfer of existing infectious pathogens to new hosts is also on the rise. When a pandemic strikes, scientists and public health officials need the ability to predict the course of a disease quickly and accurately. Public health officials rely on forecasts to create prevention and intervention strategies, such as mandatory masking or stay-at-home orders. Instead of depending solely on individual forecasts that leverage one technique or algorithm, scientists often bundle them together to create an ensemble of forecast models. These ensembles sometimes better predict long-term trends in outbreaks, hospitalizations, and deaths. However, pandemics have proven that these same ensembles fail to provide precise enough forecasts for short-term trends. From the early days of a recent pandemic, the Center for Disease Control and Prevention (CDC) and health organizations shared weekly ensemble forecasts with the public. While their ensembles had succeeded in predicting established disease trends in the past, they proved unreliable in accurately anticipating short-term changes across time and place.

It will be appreciated that embodiments herein, while described for example with respect to infection disease spread, may be applicable in domains and use cases beyond infectious disease spread without departing from the scope of the present disclosure (e.g., any rate of change per location; crime rates; migration rates; and the like).

Embodiments herein overcome the aforementioned drawbacks and more by generating high accuracy forecasts for time and location to confidently inform intervention and prevention decisions. Embodiments herein are extendable to time series forecasting models in any domain by passing a small number of operator-configurable details. Embodiments herein further provide for better performance as compared to individual models and known static ensembles.

Embodiments herein consider short-term changes and trends at a local or locality level, resulting in higher accuracy predictions. The ensembles generated and evaluated according to embodiments herein provide higher forecasting accuracy, high-confidence predictions, and support epidemic and pandemic-related decision making, resource allocation, resource planning, and other automated actions.

Embodiments herein provide users with the ability to select parameters (e.g., number of horizons, ensemble levels, performance metrics, validation/test period range, and the like) for predictive analysis dynamically, which allows for testing any short term, long term, declination, holiday season, surge period, recent period, and/or the like.

In contrast to existing dynamic ensemble techniques, embodiments herein not only consider existing weighting schemes in scheme combination vectors (e.g., also referred to herein as “Scheme x Combinations”), but also consider the top x (x<=X models in model bank) models and individual models while picking the best ensemble forecast for the optimal validation period range and ensemble level under consideration. That is, embodiments herein provide for an ensembler that can pick any individual combination, static combination, or any Scheme x Combinations. Embodiments automatically evaluate and select the best dynamic precision ensemble out of the contender individual models and all possible ensemble Schemes x Combinations at each forecast or prediction generation point. Embodiments herein further consider ensemble levels; that is, embodiments herein determine whether the best model is sought at the location level, the horizon level, or the location+horizon level, and automatically evaluate which level would provide the best results for the desired forecast.

By providing dynamic precision time series ensemble selection, embodiments herein provide technical solutions to the problem of generating accurate forecasting while the data upon which the forecasting is reliant or based remains fresh and meaningful. That is, without solutions herein, forecasts may be generated that are not accurate, and/or forecasts may be generated that, while accurate, are no longer useful by the time they are generated because the underlying data is expired, or no longer up to date.

Embodiments herein further provide for the automated initiation or performance of prediction-based actions based on predictive values generated according to the present disclosure. By automatically performing, or initiating the performance of, prediction-based actions based on the predictive values, embodiments herein ensure that the appropriate action is taken at the appropriate time, and not according to approximations.

### V. EXAMPLE SYSTEM OPERATIONS

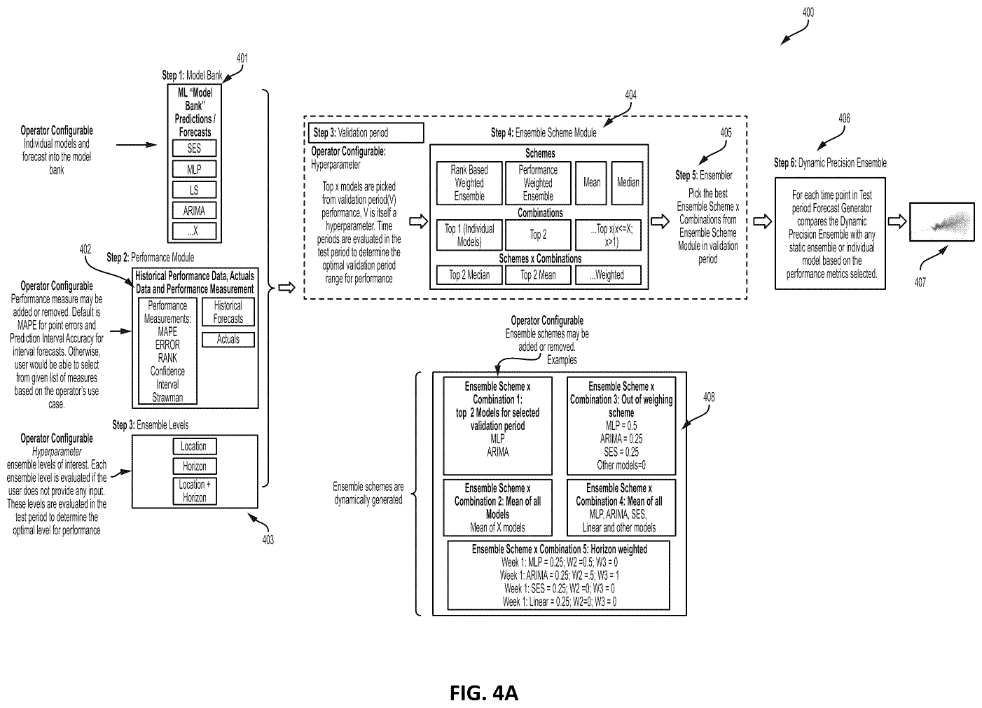

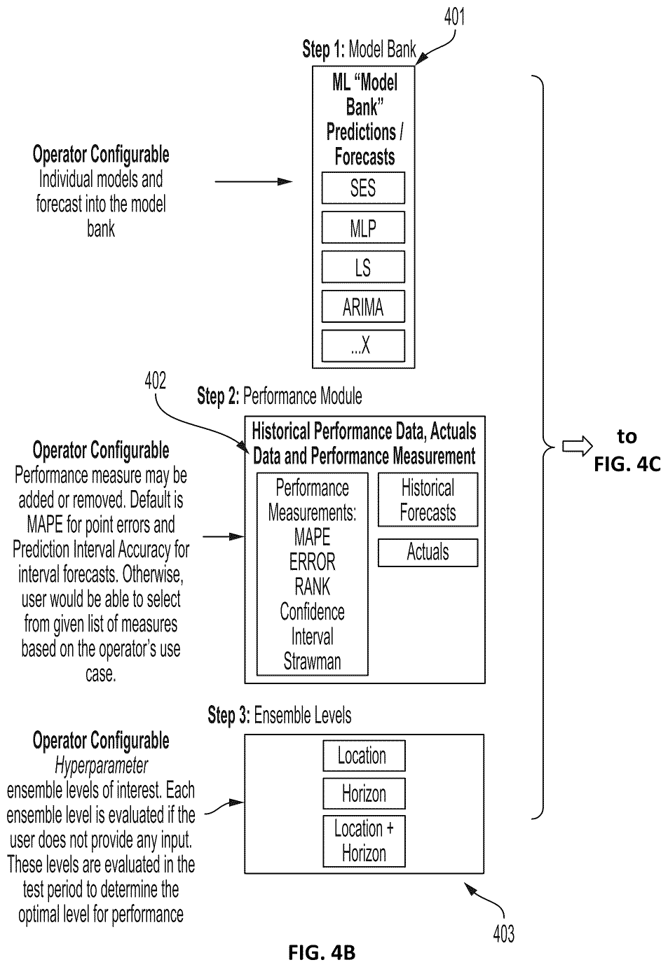

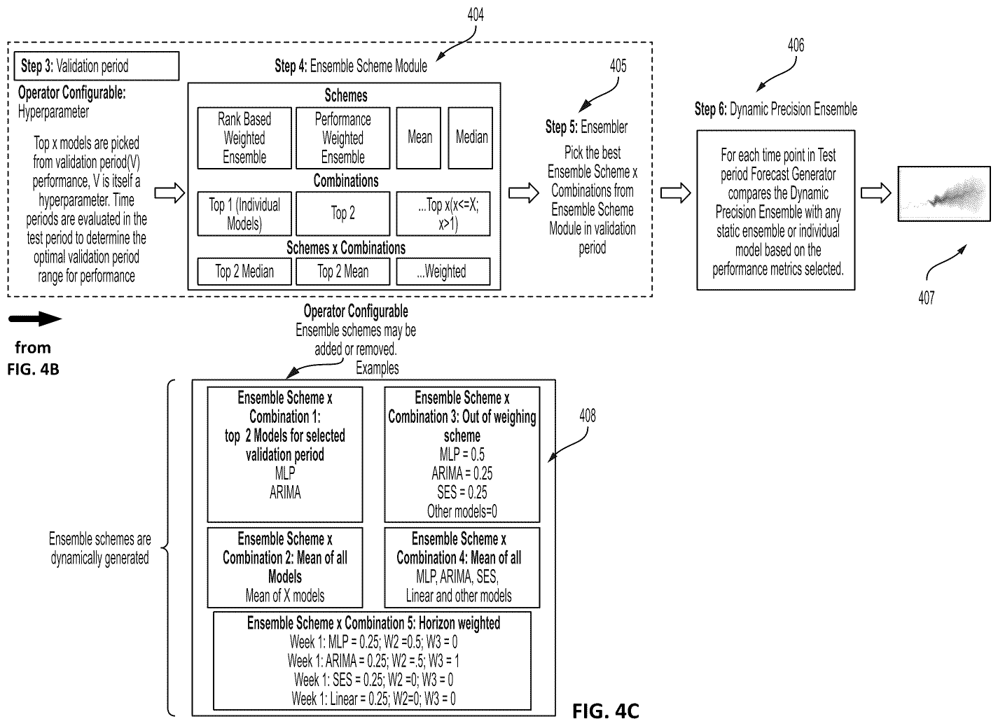

FIGS. 4A, 4B, and 4C provide an example data flow architecture in accordance with some embodiments discussed herein. An example data flow architecture, according to some embodiments, includes a model bank 401 (e.g., of multiple forecasting models), a performance module 402 (e.g., for measuring and tracking model performance using a variety of performance measures), hyperparameter selection 403, an ensemble scheme module 404 (e.g., maintaining the multiple approaches for combining model outputs; the approaches are evaluated during a selected validation period), an ensembler 405 (e.g., selects the best model or ensemble based on those evaluated during the validation period), a dynamic precision ensemble module 406 (e.g., generates ensemble output reports 407), and dynamic generation of ensemble schemes and combinations 408.

In some embodiments, the model bank 401 includes multiple machine learning models trained to predict the future risk of infectious diseases by location. It will be appreciated that forecasts and predictions of infectious disease occurrences are used interchangeably herein. Models in the model bank 401 can vary from time-series forecasting approaches (e.g., ARIMA model) to neural networks. As described above, horizons as used herein represent the time-period of the prediction relative to the current point in time. A horizon resolution may represent a week or a day or a year or other time period (e.g., minutes, hours, quarters, or other operator configurable horizon) . . . . Each time-period model generates output for the specified geographic resolution (e.g., zip code, county, state, and/or the like), for h horizons, where h represents a number of sequential periods (e.g., operator specified, pre-defined, dynamically configurable).

In some embodiments, the performance module 402 measures the performance of each model (e.g., in the model bank 401) and each ensemble combination of models (e.g., in the model bank 401) relative to the actual outcome using a variety of performance measurements. In some examples, performance measurements may include mean absolute percentage error (MAPE), mean absolute error (MAE), root mean square error (RMSE), prediction interval accuracies, and/or the like.

In some embodiments, hyperparameter selection 403 includes selection of ensemble level and validation period. An optimal ensemble level, for example one of location level, horizon level, or a combination of location and horizon level, and validation period is selected. Ensemble levels and validation periods are treated as hyperparameters, and their respective optimal values are selected before the ensemble scheme module 404 starts generating Scheme x Combinations.

In some embodiments, the ensemble scheme module 404 generates all possible schemes and all combinations 408 of the top x models (where X represents the number of models in the model bank 401 and 1≤x≤X) to pass to the ensembler 405. The top x models and best Scheme x Combination are selected from the validation period.

In some embodiments, the ensembler 405 selects the best scheme and combination based on performance measures provided by the performance module 402 and based on the optimal validation period and ensemble level. That is, ensembles of models are dynamically created for the optimal ensemble level and optimal validation period selected during test period using the different ensemble schemes defined above. The operator passes the ensemble level(s) and validation period(s) of interest. The validation periods of interest are represented as an array of time periods. Each ensemble level is evaluated if the user does not provide any input. The ensemble levels and validation periods are then evaluated in the test period to determine the optimal level for performance.

In some embodiments, a dynamic precision ensemble module 406 then generates h (e.g., where h is the number of horizons) forecasts or predictions (e.g., ensemble output reports 407) for the chosen optimal validation period and ensemble levels using the ensembler 405.

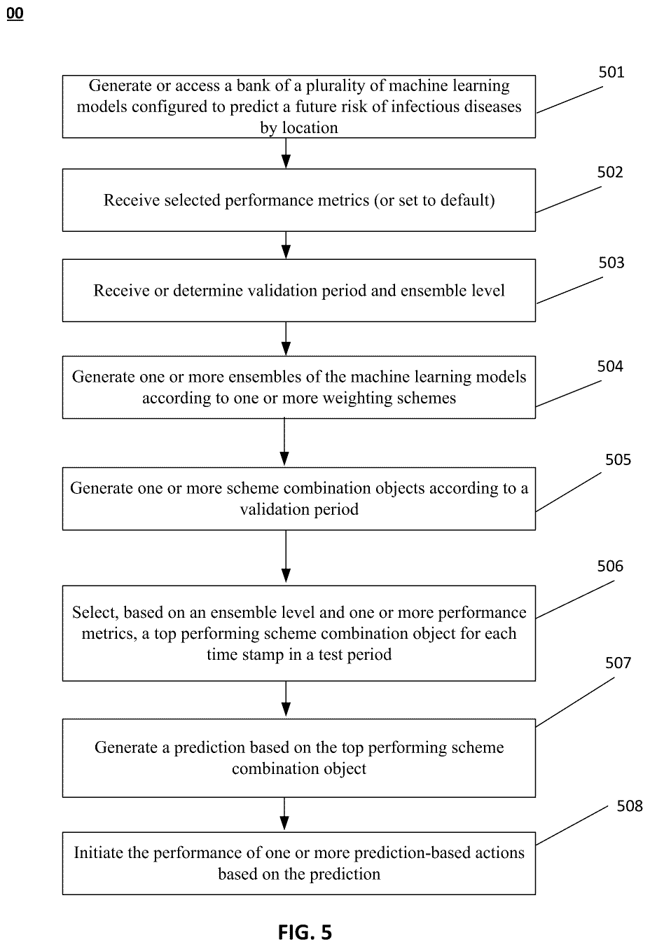

FIG. 5 provides example operations for performance in accordance with some embodiments discussed herein. Via the various steps/operations of the process 500, a predictive analysis computing entity 106 efficiently, effectively, and accurately predicts or forecasts the risk of spread of infectious disease for a particular locality. That is, the predictive analysis computing entity 106 may consider short-term changes and trends at a local or locality level, resulting in higher accuracy predictions. The ensembles generated and evaluated by the predictive analysis computing entity 106 provide higher forecasting accuracy, high-confidence predictions, and support epidemic and pandemic-related decision making, resource allocation, resource planning, and other automated actions. It will be appreciated that process 500 is depicted or described herein using examples for illustrative purposes that are not intended to be limiting. For example, The example below illustrates a dynamic ensemble on a location+horizon level (e.g., it is appreciated that dynamic ensembles are also available on a location or horizon level); also, geospatial resolution in this example is on a county level and the forecast horizon is measured in weeks (e.g., it is appreciated that other levels and horizons are available within the scope of the present disclosure).

In some embodiments the process 500 begins at step/operation 501 when the predictive analysis computing entity 106 generates, updates, or retrieves from a model bank (e.g., 401). For example, each period of time (e.g., each week), multiple models (e.g., X models where X is a number) are added to the model bank (e.g., 401) Each model generates H time-series predictions for L locations. A combination vector may be generated for each combination of predictions for the L locations.

An example combination vector X₁ L₃ H₀ represents model 1, county 3 and the forecast for the current week (e.g., week 0). Another example combination vector X₂ L₃ H₀ represents model 2, county 3 and the forecast for the current week (e.g., week 0). Another example combination vector Xₓ Lₗ Hₕ represents model x, county 1, and the forecast for week h (e.g., h or h−1 weeks from a current network time).

In some embodiments the process 500 continues at step/operation 502 when the predictive analysis computing entity 106 receives performance metrics for use in the analysis (e.g., comparing predictions against actual outcome). For example, the predictive analysis computing entity 106 may receive selections of performance metrics, including MAPE, MAE, RMSE, interval accuracy, and/or the like (e.g., it will be appreciated that other performance measures can be considered). In some embodiments, if no performance metrics are selected, the system may select default performance metrics.

Continuing the example from above, a performance measurement is denoted by Pₚ where P₁ through Pᵢ (i≤p; p=total #of performance metrics) represents the calculated metric. The example below displays the performance measures P for individual model X₁ (TABLE 1), individual model X₂ (TABLE 2) and for an ensemble Scheme x Combination Eₑ (e.g., mean of top x models) (TABLE 3) on Location+Horizon ensemble level for 4 predictive horizons for county 3.

TABLE 1
Model X₁ output

| Forecast | | | | | |
|---|---|---|---|---|---|
| X₁L₃ | P₁ _{MAPE} | P₂ _{Interval Accuracy} | P₃ _{MAE} | P₄ _{RMSE} | . . . Pₚ |
| X₁ L₃ H₀ | p₁ | p₂ | p₃ | p₄ | . . . pₚ |
| X₁ L₃ H₁ | p₁ | p₂ | p₃ | p₄ | . . . pₚ |
| X₁ L₃ H₂ | p₁ | p₂ | p₃ | p₄ | . . . pₚ |
| X₁ L₃ H₃ | p₁ | p₂ | p₃ | p₄ | . . . pₚ |

TABLE 2
Model X₂ output

| Forecast | | | | | |
|---|---|---|---|---|---|
| X₂L₃ | P₁ _{MAPE} | P₂ _{Interval Accuracy} | P₃ _{MAE} | P₄ _{RMSE} | . . . Pₚ |
| X₂ L₃ H₀ | p₁ | p₂ | p₃ | p₄ | . . . pₚ |
| X₂ L₃ H₁ | p₁ | p₂ | p₃ | p₄ | . . . pₚ |
| X₂ L₃ H₂ | p₁ | p₂ | p₃ | p₄ | . . . pₚ |
| X₂ L₃ H₃ | p₁ | p₂ | p₃ | p₄ | . . . pₚ |

TABLE 3
Model Eₑ output

| Forecast | | | | | |
|---|---|---|---|---|---|
| EₑL₃ | P₁ _{MAPE} | P₂ _{Interval Accuracy} | P₃ _{MAE} | P₄ _{RMSE} | . . . Pₚ |
| Eₑ L₃ H₀ | p₁ | p₂ | p₃ | p₄ | . . . pₚ |
| Eₑ L₃ H₁ | p₁ | p₂ | p₃ | p₄ | . . . pₚ |
| Eₑ L₃ H₂ | p₁ | p₂ | p₃ | p₄ | . . . pₚ |
| Eₑ L₃ H₃ | p₁ | p₂ | p₃ | p₄ | . . . pₚ |

The tables below depict performance metrics for the same scenario as above but with Location ensemble level, therefore only one value would exist for each error metric.

TABLE 4
Model X₁ Output

| Forecast | P₁ _{MAPE} | P₂ _{Interval Accuracy} | P₃ _{MAE} | P₄ _{RMSE} | . . . Pₚ |
|---|---|---|---|---|---|
| X₁L₃ | p₁ | p₂ | p₃ | p₄ | . . . pₚ |

TABLE 5
Model X₂ Output

| Forecast | P₁ _{MAPE} | P₂ _{Interval Accuracy} | P₃ _{MAE} | P₄ _{RMSE} | . . . Pₚ |
|---|---|---|---|---|---|
| X₂L₃ | p₁ | p₂ | p₃ | p₄ | . . . pₚ |

TABLE 6
Model Eₑ Output

| Forecast | P₁ _{MAPE} | P₂ _{Interval Accuracy} | P₃ _{MAE} | P₄ _{RMSE} | . . . Pₚ |
|---|---|---|---|---|---|
| EₑL₃ | p₁ | p₂ | p₃ | p₄ | . . . pₚ |

In some embodiments, the iteration history for all models is constructed and updated after each forecast generation. Accordingly, more updated performance metrics are available after each forecast generation. The selection of performance measure(s) for the ensemble is configurable, and/or may be selected out of a dynamic list of error and accuracy metrics.

In some embodiments the process 500 continues at step/operation 503 when the predictive analysis computing entity 106 selects or identifies an ensemble level and validation period. For example, the predictive analysis computing entity 106 treats ensemble levels and validation periods as hyperparameters and selects their respective optimal values before the ensemble scheme module starts generating Scheme x Combinations. In some embodiments, the ensemble level and validation period are user configurable.

In some embodiments the process 500 continues at step/operation 504 when the predictive analysis computing entity 106 generates ensembles according to one or more schemes. For example, the predictive analysis computing entity 106 may generate ensembles according to mean, median, performance-based weights, rank-based weights, and/or the like. In some embodiments, static weights are assigned to each model outcome. Static weights may be generated in several ways; for example, according to trial period performance (e.g., linear regression of all models based on performance during trial periods) or median/mean of all contributing models' outputs. Rank weights may be applied according to a rank based on performance (e.g., combinations of top 1, . . . , x models).

By way of example, continuing from the example above, MAPE is used herein as an example performance evaluation metric. In TABLE 7, the models with the best MAPE measures at horizons 0, 1, 2 and 3 are E1, X2, E4 and X6 respectively, where X represents an individual model and E represents any ensemble Scheme x Combination. The second best for horizons 0, 1, 2 and 3 are the models numbered X4, E1, E5 and X3, respectively. Through this approach, the top performing model/ensemble for each horizon is shown below for county 3 when the ensemble level is location+horizon (e.g., the predictive analysis computing entity 106 searches for the best ensemble for each location and horizon combination).

TABLE 7
Models 1, 2, 3, 4 MAPE Performance for County 3 for 4 Horizons

| | Horizon 0 P₁ | Horizon 1 P₁ | Horizon 2 P₁ | Horizon 3 P₁ |
|---|---|---|---|---|
| Rank | _{MAPE performance} | _{MAPE performance} | _{MAPE performance} | _{MAPE performance} |
| 1 | E₁ L₃ H₀ | X₂ L₃ H₁ | E₄ L₃ H₂ | X₆ L₃ H₃ |
| 2 | X₄ L₃ H₀ | E₁ L₃ H₁ | E₅ L₃ H₂ | X₃ L₃ H₃ |
| 3 | X₆ L₃ H₀ | X₅ L₃ H₁ | E₁ L₃ H₂ | E₂ L₃ H₃ |
| 4 | E₂ L₃ H₀ | E₃ L₃ H₁ | X₂ L₃ H₂ | X₁ L₃ H₃ |

It will be appreciated that performance testing of models during risk surges, declination, and flat periods of an epidemic provide data for the best and worst performing models during these periods (e.g., can be passed as operator defined validation period range). Thus, if the current time period is experiencing a surge and model X₄L₃ performed best for all time horizons during a surge, the dynamic precision ensemble will give the highest weight to X₄L₃ or can pick this individual model if it performs better than any of the precision ensemble combinations.

In some embodiments the process 500 continues at step/operation 505 when the predictive analysis computing entity 106 generates multiple ensembles using pairs of combinations and schemes for the optimal validation period.

In some embodiments the process 500 continues at step/operation 506 when the predictive analysis computing entity 106 selects the best ensemble out of the Schemes x Combinations ensembles for a selected ensemble level at every time point (e.g., time stamp) in the test period and generates dynamic precision ensemble forecasts, reports, and visualization. The selection of the best ensemble may be based on the performance metrics selected in step 502.

In some embodiments the process 500 continues at step/operation 507 when the predictive analysis computing entity 106 measures performance by comparing the predicted dynamic precision ensemble values from step/operation 506 to actual outcomes during the defined test period. For example, the predictive analysis computing entity tests the performance of models during risk surges, declination, and flat periods of an epidemic and provides data for the best and worst performing models during these periods (e.g., which can be passed as operator defined validation period range). Thus, if, for example, the current time-period is experiencing a surge and model X₄L₃ performed best for all time horizons during a surge, the dynamic precision ensemble will give the highest weight to X₄L₃ or can pick this individual model if it performs better than any of the precision ensemble combinations.

In some embodiments the process 500 continues at step/operation 508 when the predictive analysis computing entity 106 initiates the performance of, or performs, one or more prediction-based action based at least in part on the dynamic precision forecasts and/or the performance of the predicted dynamic precision ensemble values. In some embodiments, the prediction-based actions may include automated updates to guidance (e.g., CDC guidance), automated updates to a public health response, automated updates to medication distributions, automated updates to travel restrictions, automated alerts, automated instructions to medication delivery devices, automated adjustments to medical equipment, automated adjustments to allocations of medical, computing, hospital, facility, and/or human resources. Further, such automated actions may include automated physician notification actions, automated patient notification actions, automated appointment scheduling actions, automated prescription recommendation actions, automated drug prescription generation actions, automated implementation of precautionary actions, automated record updating actions, automated datastore updating actions, automated hospital preparation actions, automated workforce management operational management actions, automated server load balancing actions, automated resource allocation actions, automated call center preparation actions, automated hospital preparation actions, automated pricing actions, automated plan update actions, automated alert generation actions, generating a diagnostic report, generating action scripts, generating one or more electronic communications, and/or the like. The one or more prediction-based actions may further include displaying visual renderings of the aforementioned examples of prediction-based actions in addition to values, charts, and representations associated with the prediction output using a prediction output user interface.

As discussed herein, some techniques of the present disclosure enable the generation of new machine learning models with parameters specifically trained and tailored to perform one or more prediction-based actions to achieve real-world affects. The machine learning models of the present disclosure may be used, applied, and/or otherwise leveraged to generate predictions. These predictions may be leveraged to initiate the performance of various computing tasks that improve the performance of a computing system (e.g., a computer itself, etc.) with respect to various prediction-based actions performed by the computing system.

In some examples, the computing tasks may include prediction-based actions that may be based on a prediction domain. A prediction domain may include any environment in which computing systems may be applied to achieve real-word insights, such as predictions, and initiate the performance of computing tasks, such as prediction-based actions, to act on the real-world insights. These prediction-based actions may cause real-world changes, for example, by controlling a hardware component, providing targeted alerts, automatically allocating computing or human resources, and/or the like.

Examples of prediction domains may include financial systems, clinical systems, autonomous systems, robotic systems, and/or the like. Prediction-based actions in such domains may include the initiation of automated instructions across and between devices, automated notifications, automated scheduling operations, automated precautionary actions, automated security actions, automated data processing actions, automated server load balancing actions, automated computing resource allocation actions, automated adjustments to computing and/or human resource management, and/or the like.

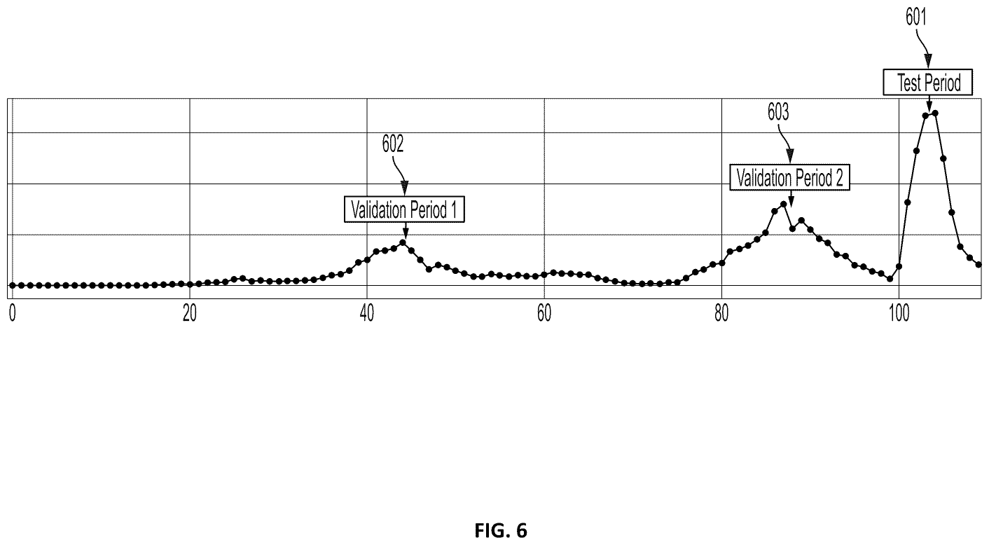

FIG. 6 provides examples of operator configurable validation periods associated with some embodiments discussed herein. In FIG. 6, a predictive analysis computing entity 106 may be configured to determine (e.g., or receive input designating) the optimal validation period to be used to a dynamic precision ensemble according to historic surges. In such an example, a Validation Period 1 (e.g., 602), Validation Period 2 (e.g., 603), and another validation period (e.g., test period 601) can be passed to the predictive analysis computing entity 106 so that the best Scheme x Combination can be selected, where the best Scheme x Combination is the ensemble that performed best during previous surges. Accordingly, based on the example in FIG. 6, the surge or validation period 601 is selected as the test period 601 in the analysis.

### VI. CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### VII. EXAMPLES

Example 1. A system comprising memory and one or more processors communicatively coupled to the memory, the one or more processors configured to generate one or more ensembles of machine learning models according to one or more weighting schemes, wherein the machine learning models are selected from a plurality of machine learning models configured to predict a future risk of infectious diseases by location; generate one or more scheme combination objects according to a validation period, wherein the one or more scheme combination objects comprise one or more machine learning models to which a particular weighting scheme has been applied; select, based on an ensemble level and one or more performance metrics, a top performing scheme combination object for each time stamp in a test period; generate a prediction based on the top performing scheme combination object; and initiate the performance of one or more prediction-based actions based on the prediction.

Example 2. A system in accordance with the foregoing Example, wherein the one or more processors are further configured to retrieve the plurality of machine learning models; and generate the one or more performance metrics associated with each machine learning model of the plurality of machine learning models and one or more combinations of two or more of the plurality of machine learning models.

Example 3. A system in accordance with any of the foregoing Examples, wherein the one or more performance metrics comprise one or more of mean absolute percentage error (MAPE), root mean square error (RMSE), mean absolute error (MAE), or interval accuracies.

Example 4. A system in accordance with any of the foregoing Examples, wherein one or more of the plurality of machine learning models comprises a time series linear regression model.

Example 5. A system in accordance with any of the foregoing Examples, wherein the one or more processors are further configured to: receive or determine one or more of the ensemble level or the validation period.

Example 6. A system in accordance with any of the foregoing Examples, wherein determining the ensemble level is based on evaluating one or more performance metrics for each ensemble level of a plurality of ensemble levels in the test period.

Example 7. A system in accordance with any of the foregoing Examples, wherein determining the validation period is based on evaluating one or more performance metrics for each available validation period of a plurality of validation periods in the test period.

Example 8. A system in accordance with any of the foregoing Examples, wherein the ensemble level comprises at least one of horizon, location, or location+horizon.

Example 9. A system in accordance with any of the foregoing Examples, wherein an ensemble comprises two or more machine learning models of the plurality of machine learning models.

Example 10. A system in accordance with any of the foregoing Examples, wherein a combination vector represents a combination of predictions for locations in a geospatial region having historical time-series data.

Example 11. A system in accordance with any of the foregoing Examples, wherein a scheme combination object represents at least one of an individual machine learning model or an ensemble of machine learning models to which a particular weighting scheme is applied.

Example 12. A system in accordance with any of the foregoing Examples, wherein a horizon comprises network time points at which predictions are performed using one or more of the plurality of machine learning models.

Example 13. A system in accordance with any of the foregoing Examples, wherein the horizon comprises at least one of seconds, minutes, hours, days, weeks, months, quarters, years, or a received horizon selection.

Example 14. A system in accordance with any of the foregoing Examples, wherein the one or more prediction-based actions comprise at least one of automated updates to guidance, automated updates to a public health response, automated updates to medication distributions, automated updates to travel restrictions, automated alerts, automated instructions to medication delivery devices, automated adjustments to medical equipment, automated adjustments to allocations of medical, computing, hospital, facility, and/or human resources, automated physician notification actions, automated patient notification actions, automated appointment scheduling actions, automated prescription recommendation actions, automated drug prescription generation actions, automated implementation of precautionary actions, automated record updating actions, automated datastore updating actions, automated hospital preparation actions, automated workforce management operational management actions, automated server load balancing actions, automated resource allocation actions, automated call center preparation actions, automated hospital preparation actions, automated pricing actions, automated plan update actions, automated alert generation actions, generating a diagnostic report, generating action scripts, or generating one or more electronic communications.

Example 15. A system in accordance with any of the foregoing Examples, wherein the one or more processors are further configured to: cause rendering of a graphical representation of the prediction via a user interface or display device.

Example 16. A system in accordance with any of the foregoing Examples, wherein the validation period is a point of network time or a duration of network time during which one or more of the plurality of machine learning models, ensembles, or scheme combination objects are determined to have best performed for the ensemble level.

Example 17. A system in accordance with any of the foregoing Examples, wherein the test period is a point of network time or a duration of network time during which one or more performance metrics of a dynamic precision ensemble output is compared to one or more performance metrics of any individual machine learning model output or static ensemble output.

Example 18. A system in accordance with any of the foregoing Examples, wherein the prediction represents a likelihood of infectious disease spread.

Example 19. One or more non-transitory computer-readable storage media including instructions that, when executed by one or more processors, cause the one or more processors to: generate one or more ensembles of machine learning models according to one or more weighting schemes, wherein the machine learning models are selected from a plurality of machine learning models configured to predict a future risk of infectious diseases by location; generate one or more scheme combination objects according to a validation period, wherein the one or more scheme combination objects comprise one or more machine learning models to which a particular weighting scheme has been applied; select, based on an ensemble level and one or more performance metrics, a top performing scheme combination object for each time stamp in a test period; generate a prediction based on the top performing scheme combination object; and initiate the performance of one or more prediction-based actions based on the prediction.

Example 20. A computer-implemented method comprising: generating, by one or more processors, one or more ensembles of machine learning models according to one or more weighting schemes, wherein the machine learning models are selected from a plurality of machine learning models configured to predict a future risk of infectious diseases by location; generating, by the one or more processors, one or more scheme combination objects according to a validation period, wherein the one or more scheme combination objects comprise one or more machine learning models to which a particular weighting scheme has been applied; selecting, by the one or more processors and based on an ensemble level and one or more performance metrics, a top performing scheme combination object for each time stamp in a test period; generating, by the one or more processors, a prediction based on the top performing scheme combination object; and initiating, by the one or more processors, the performance of one or more prediction-based actions based on the prediction.

1. A system comprising memory and one or more processors communicatively coupled to the memory, the one or more processors configured to:
generate one or more ensembles of machine learning models according to one or more weighting schemes, wherein the machine learning models are selected from a plurality of machine learning models configured to predict a future risk of infectious diseases by location;
generate one or more scheme combination objects according to a validation period, wherein the one or more scheme combination objects comprise one or more machine learning models to which a particular weighting scheme has been applied;
select, based on an ensemble level and one or more performance metrics, a top performing scheme combination object for each time stamp in a test period;
generate a prediction based on the top performing scheme combination object; and
initiate the performance of one or more prediction-based actions based on the prediction.

2. The system of claim 1, wherein the one or more processors are further configured to:
retrieve the plurality of machine learning models; and
generate the one or more performance metrics associated with each machine learning model of the plurality of machine learning models and one or more combinations of two or more of the plurality of machine learning models.

3. The system of claim 1, wherein the one or more performance metrics comprise one or more of mean absolute percentage error (MAPE), root mean square error (RMSE), mean absolute error (MAE), or interval accuracies.

4. The system of claim 1, wherein one or more of the plurality of machine learning models comprises a time series linear regression model.

5. The system of claim 1, wherein the one or more processors are further configured to:
receive or determine one or more of the ensemble level or the validation period.

6. The system of claim 5, wherein determining the ensemble level is based on evaluating one or more performance metrics for each ensemble level of a plurality of ensemble levels in the test period.

7. The system of claim 5, wherein determining the validation period is based on evaluating one or more performance metrics for each available validation period of a plurality of validation periods in the test period.

8. The system of claim 1, wherein the ensemble level comprises at least one of horizon, location, or location+horizon.

9. The system of claim 1, wherein an ensemble comprises two or more machine learning models of the plurality of machine learning models.

10. The system of claim 1, wherein a combination vector represents a combination of predictions for locations in a geospatial region having historical time-series data.

11. The system of claim 1, wherein a scheme combination object represents at least one of an individual machine learning model or an ensemble of machine learning models to which a particular weighting scheme is applied.

12. The system of claim 1, wherein a horizon comprises network time points at which predictions are performed using one or more of the plurality of machine learning models.

13. The system of claim 12, wherein the horizon comprises at least one of seconds, minutes, hours, days, weeks, months, quarters, years, or a received horizon selection.

14. The system of claim 1, wherein the one or more prediction-based actions comprise at least one of automated updates to guidance, automated updates to a public health response, automated updates to medication distributions, automated updates to travel restrictions, automated alerts, automated instructions to medication delivery devices, automated adjustments to medical equipment, automated adjustments to allocations of medical, computing, hospital, facility, and/or human resources, automated physician notification actions, automated patient notification actions, automated appointment scheduling actions, automated prescription recommendation actions, automated drug prescription generation actions, automated implementation of precautionary actions, automated record updating actions, automated datastore updating actions, automated hospital preparation actions, automated workforce management operational management actions, automated server load balancing actions, automated resource allocation actions, automated call center preparation actions, automated hospital preparation actions, automated pricing actions, automated plan update actions, automated alert generation actions, generating a diagnostic report, generating action scripts, or generating one or more electronic communications.

15. The system of claim 1, wherein the one or more processors are further configured to:
cause rendering of a graphical representation of the prediction via a user interface or display device.

16. The system of claim 1, wherein the validation period is a point of network time or a duration of network time during which one or more of the plurality of machine learning models, ensembles, or scheme combination objects are determined to have best performed for the ensemble level.

17. The system of claim 1, wherein the test period is a point of network time or a duration of network time during which one or more performance metrics of a dynamic precision ensemble output is compared to one or more performance metrics of any individual machine learning model output or static ensemble output.

18. The system of claim 1, wherein the prediction represents a likelihood of infectious disease spread.

19. One or more non-transitory computer-readable storage media including instructions that, when executed by one or more processors, cause the one or more processors to:
generate one or more ensembles of machine learning models according to one or more weighting schemes, wherein the machine learning models are selected from a plurality of machine learning models configured to predict a future risk of infectious diseases by location;
generate one or more scheme combination objects according to a validation period, wherein the one or more scheme combination objects comprise one or more machine learning models to which a particular weighting scheme has been applied;
select, based on an ensemble level and one or more performance metrics, a top performing scheme combination object for each time stamp in a test period;
generate a prediction based on the top performing scheme combination object; and
initiate the performance of one or more prediction-based actions based on the prediction.

20. A computer-implemented method comprising:
generating, by one or more processors, one or more ensembles of machine learning models according to one or more weighting schemes, wherein the machine learning models are selected from a plurality of machine learning models configured to predict a future risk of infectious diseases by location;
generating, by the one or more processors, one or more scheme combination objects according to a validation period, wherein the one or more scheme combination objects comprise one or more machine learning models to which a particular weighting scheme has been applied;
selecting, by the one or more processors and based on an ensemble level and one or more performance metrics, a top performing scheme combination object for each time stamp in a test period;
generating, by the one or more processors, a prediction based on the top performing scheme combination object; and
initiating, by the one or more processors, the performance of one or more prediction-based actions based on the prediction.
